(19) United States
(12) Patent Application Publication
(10) Pub. No.: US 2025/0155306 A1
(43) Pub. Date: May 15, 2025

(54) PULSE WAVE SENSOR

(71) Applicant: MINEBEA MITSUMI Inc., Nagano (JP)

(72) Inventors: Keisuke KOUDA, Tokyo (JP); Makoto KITAZUME, Tokyo (JP); Shigenori INAMOTO, Tokyo (JP); Shigeyuki ADACHI, Nagano (JP); Toshiaki ASAKAWA, Nagano (JP); Satoshi SATO, Nagano (JP); Atsushi KITAMURA, Nagano (JP)

(21) Appl. No.: 18/841,102

(22) PCT Filed: Feb. 28, 2023
(86) PCT No.: PCT/JP2023/007260

§ 371 Date: Aug. 23, 2024

(30) Foreign Application Priority Data
Mar. 1, 2022 (JP) 2022-030926
Aug. 16, 2022 (JP) 2022-129701
Jan. 31, 2023 (JP) 2023-013171

(51) Int. Cl. G01L 9/00; G01L 9/16

(52) U.S. Cl. CPC G01L 9/0052; G01L 9/16

(57) ABSTRACT

The present pulse wave sensor includes a strain generating body including a plurality of slits each having an elongated shape and bending in a same direction, and a strain gauge provided on the strain generating body and including a Cr mixed phase film as a resistor. The plurality of slits are varied in a distance from a center of gravity of the strain generating body to one-side ends of the slits and in a distance from the center of gravity of the strain generating body to the other-side ends of the slits. The pulse wave sensor is configured to detect a pulse wave based on a change in a resistance value of the resistor in response to a deformation of the strain generating body.

### TECHNICAL FIELD

The present invention relates to a pulse wave sensor.

### BACKGROUND ART

A pulse wave sensor for detecting arterial pulses generated when the heart pumps blood is known. An example of the pulse wave sensor is a pulse wave sensor provided with a pressure receiving plate as a strain generating body supported in a deflective manner by an action of an external force and a piezoelectric conversion element for converting the deflection of the pressure receiving plate into an electric signal. The pulse wave sensor has a dome-like shape in which a deflective region of the pressure receiving plate becomes a convex curved surface outward, and a pressure detecting element is provided on the inner surface of the top of the pressure receiving plate as the piezoelectric conversion element (for example, Patent Document 1).

### CITATION LIST

### Patent Literature

[Patent Document 1] Japanese Unexamined Patent Application Publication No. 2002-78689

### SUMMARY OF THE INVENTION

### Technical Problem

Although a pulse wave sensor needs to detect a minute signal, it is difficult for the strain generating body structure of an existing pulse wave sensor to generate a sufficient amount of a strain.

The present invention was made in view of the above point, and an object of the present invention is to provide a pulse wave sensor including a strain generating body having an easy-to-strain structure.

### Solution to the Problem

The present pulse wave sensor includes: a strain generating body including a plurality of slits each having an elongated shape and bending in a same direction; and a strain gauge provided on the strain generating body and including a Cr mixed phase film as a resistor, wherein the plurality of slits are varied in a distance from a center of gravity of the strain generating body to one-side ends of the slits and in a distance from the center of gravity of the strain generating body to the other-side ends of the slits, and the pulse wave sensor is configured to detect a pulse wave based on a change in a resistance value of the resistor in response to a deformation of the strain generating body.

### Advantageous Effects of the Invention

The disclosed technique can provide a pulse wave sensor including a strain generating body having an easy-to-strain structure.

### BRIEF DESCRIPTION OF THE DRAWINGS

FIG. 1 is a perspective view illustrating a pulse wave sensor according to a first embodiment.

FIG. 2 is a plan view illustrating the pulse wave sensor according to the first embodiment.

FIG. 3 is a bottom view illustrating the pulse wave sensor according to the first embodiment.

FIG. 4 is a cross-sectional view illustrating the pulse wave sensor according to the first embodiment.

FIG. 5 is a view illustrating a simulation result.

FIG. 6 is a plan view illustrating a strain gauge according to the first embodiment.

FIG. 7 is a cross-sectional view illustrating the strain gauge according to the first embodiment.

FIG. 8 is a cross-sectional view (part 1) illustrating a pulse wave sensor according to a modified example of the first embodiment.

FIG. 9 is a cross-sectional view (part 2) illustrating a pulse wave sensor according a modified example of the first embodiment.

FIG. 10 includes a plan view and a cross-sectional view illustrating an example of a detecting element included in a strain gauge according to a second embodiment.

FIG. 11 includes a perspective view, a plan view, and a cross-sectional view illustrating an example of a detecting element included in a strain gauge according to a third embodiment.

FIG. 12 includes a perspective view, a plan view, and a cross-sectional view illustrating another example of the detecting element included in the strain gauge according to the third embodiment.

FIG. 13 includes a perspective view, a plan view, and a cross-sectional view illustrating yet another example of the detecting element included in the strain gauge according to the third embodiment.

### DETAILED DESCRIPTION OF THE INVENTION

Embodiments for carrying out the invention will be described below with reference to the drawings. In the drawings, the same components may be denoted by the same reference numerals. In the drawings, there are cases where an X-direction, a Y-direction, and a Z-direction that are orthogonal to each other are defined. In such cases, a starting point (base) side of an arrow in the X-direction may be referred to as an X− side, and an ending point (arrow head) side of the arrow in the X-direction may be referred to as an X+ side. The same applies to the Y-direction and the Z-direction. In the drawings, description of the same components as those already described may be omitted.

### First Embodiment

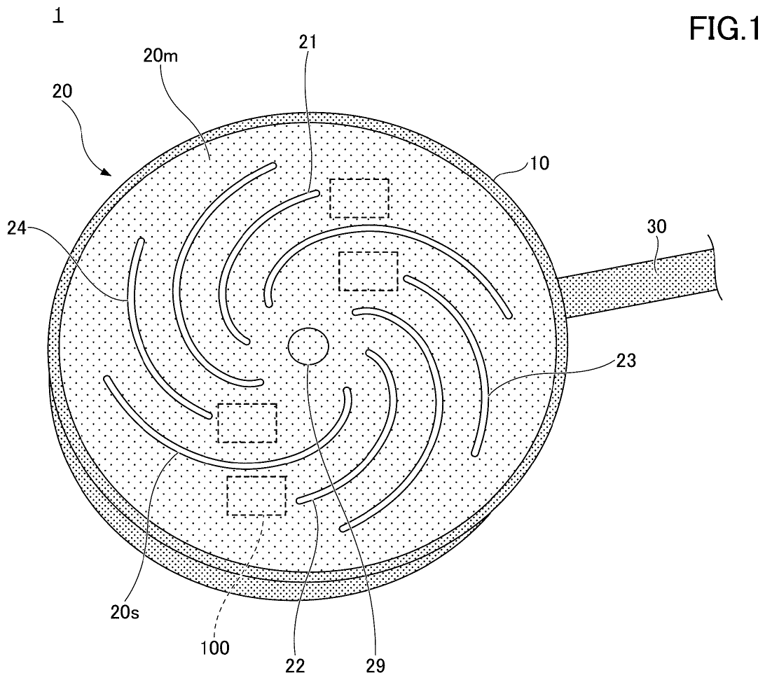

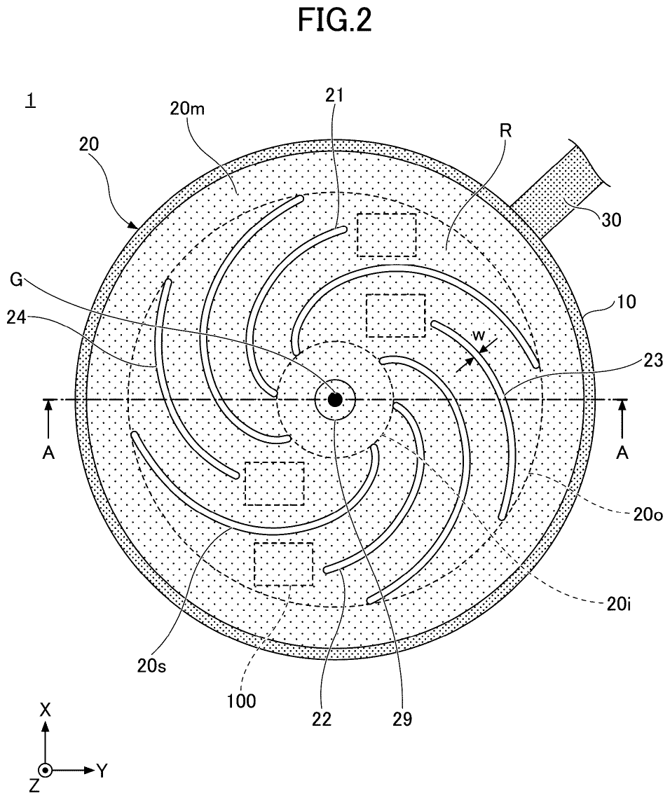

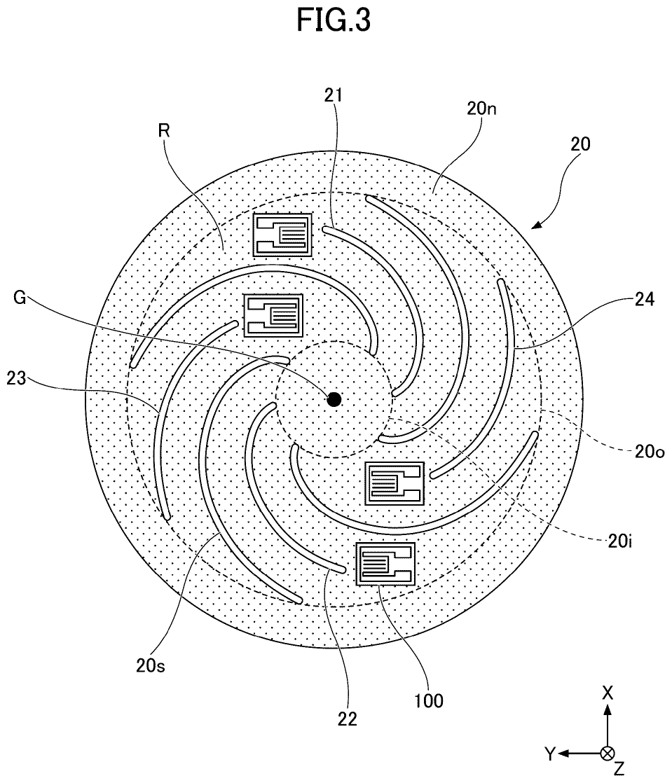

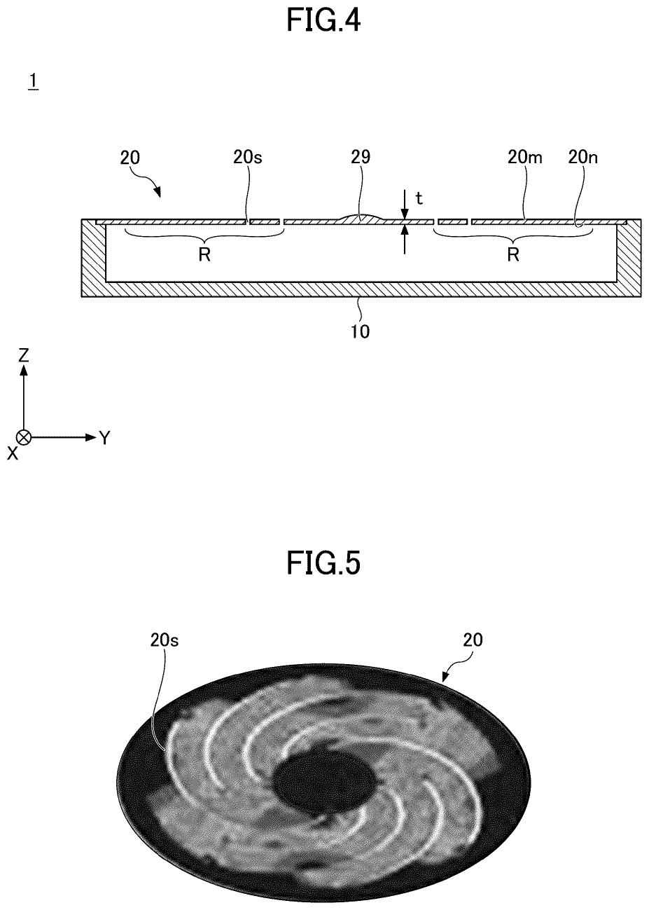

FIG. 1 is a perspective view illustrating a pulse wave sensor according to a first embodiment. FIG. 2 is a plan view illustrating the pulse wave sensor according to the first embodiment. FIG. 3 is a bottom view illustrating the pulse wave sensor according to the first embodiment. FIG. 4 is a cross-sectional view illustrating the pulse wave sensor according to the first embodiment, and illustrates a cross-section taken along a line A-A of FIG. 2. Here, a plan view is a view seen from a side to which a load portion 29 projects, and a bottom view is a view seen from a side on which strain gauges 100 are provided.

With reference to FIG. 1 to FIG. 4, a pulse wave sensor 1 includes a housing 10, a strain generating body 20, a wire rod 30, and strain gauges 100.

In the description of the pulse wave sensor 1 with reference to FIG. 1 to FIG. 4, for the sake of expediency, in the pulse wave sensor 1, a side of the strain generating body 20 having a surface that is to contact the radial artery of a subject is referred to as the “upper side”, and the side thereof having the opposite surface is referred to as the “lower side”. A surface of each part positioned on the upper side is referred to as the “upper surface”, and a surface of each part positioned on the lower side is referred to as the “lower side”. However, the pulse wave sensor 1 can be used upside down. Moreover, the pulse wave sensor 1 can be positioned at any desirably selected angle. Moreover, seeing in a plan view means seeing an object in a direction normal to an upper surface 20m of the strain generating body 20 from the upper side to the lower side. A planar shape refers to the shape of the object viewed in this normal direction.

The housing 10 holds the strain generating body 20. For example, the housing 10 has a hollow cylindrical shape, and the lower surface side is closed and the upper surface side is opened. The housing 10 can be formed of, for example, metal, resin, or the like. So as to close the opening on the upper surface side of the housing 10, a substantially disk-shaped strain generating body 20 is fixed by an adhesive or the like.

The strain generating body 20 is used with the upper surface 20m being made to contact the radial artery of a subject. In response to a load being applied to the strain generating body 20 in accordance with a pulse wave of the subject, the strain generating body 20 elastically deforms in accordance with the magnitude of the load.

The strain generating body 20 has, for example, a flat plate shape. The strain generating body 20 is made of, for example, a metal. Examples of the metal of which the strain generating body 20 is made include stainless steel, phosphor bronze, aluminum, and the like. Among these, it is preferable to use stainless steel in terms of corrosion resistance and a high strain. The strain generating body 20 may be formed by, for example, a pressing process and the like. The strain generating body 20 has, for example, a two-fold symmetrical shape in a plan view.

The thickness t of the strain generating body 20 is preferably 0.02 mm or greater and 0.2 mm or less. The smaller the thickness t of the strain generating body 20, the higher the sensitivity whereas the lower the rigidity. The larger the thickness t of the strain generating body 20, the higher the rigidity whereas the lower the sensitivity. By adjusting the thickness t of the strain generating body 20 to 0.02 mm or greater and 0.2 mm or less, it is possible to satisfy both rigidity and sensitivity.

The shape of the strain generating body 20 is any desirably selected shape such as a circular shape, an elliptical shape, a rectangular shape, and the like. It is preferable that the strain generating body 20 has a circular shape, because it is possible to provide slits 20s described below in the strain generating body 20 and to reduce the size of the entire strain generating body 20. The following description will be based on a case where the shape of the strain generating body 20 is a circular shape.

The strain generating body 20 includes a plurality of slits 20s each having an elongated shape and bending in the same direction. The width of the slits 20s may be constant at the respective positions in the longer direction of the slits, but does not need to be constant. Here, the elongated shape means a shape having a width-to-length ratio of 1:3 or greater. The width is defined by the average width of the respective positions in the longer direction. The length is defined by the length of a line that connects the positions that are at the half the width at the respective positions in the longer direction.

Bending in the same direction means the slits 20s bending to be convex toward points that are defined as points on the circumference of a second imaginary circle 20o (described below) that are the closest to the longer-direction centers of the slits 20s.

The plurality of slits 20s are varied in the distance from the center of gravity G of the strain generating body 20 to one-side ends of the slits 20s and in the distance from the center of gravity G of the strain generating body 20 to the other-side ends of the slits. That is, the slits 20s are not shapes that are along the circumference of an imaginary circle centered on the center of gravity G of the strain generating body 20 and having a desirably selected radius. In the strain generating body 20, the regions sandwiched between the slits 20s function as beams. By providing the plurality of slits 20s having such a shape, it is possible to realize the pulse wave sensor 1 including a strain generating body 20 having an easy-to-strain structure.

The center of gravity G here means the center of gravity in a plan view, i.e., the center of gravity of a plane figure, for which thickness is ignored. For example, since the planar shape of the strain generating body 20 illustrated in FIG. 1 to FIG. 4 is a circular shape, the center of gravity G of the strain generating body 20 corresponds to the center of the circle that constitutes the strain generating body 20.

In FIG. 2, 20i denotes a first imaginary circle centered on the center of gravity G of the strain generating body 20, and 20o represents a second imaginary circle centered on the center of gravity G of the strain generating body 20. The second imaginary circle 20o has a diameter larger than that of the first imaginary circle 20i. It is preferable that the plurality of slits 20s are positioned in a region R between the circumference of the first imaginary circle 20i and the circumference of the second imaginary circle 20o.

By providing the slits 20s in the region R of the strain generating body 20 excluding the center side and the peripheral side, it is possible to ensure the strain generating body 20 the required rigidity on the whole, and to make the region R in which the slits 20s are provided easily deformable. For example, the diameter of the first imaginary circle 20i may be approximately from ⅕ through ¼ the diameter of the strain generating body 20. The diameter of the second imaginary circle 20o may be approximately from ¾ through ⅘ the diameter of the strain generating body 20.

It is preferable that the plurality of slits 20s include two or more slits of which the one-side ends are positioned on the circumference of the first imaginary circle 20i and the other-side ends are positioned on the circumference of the second imaginary circle 20o. This can make the slits 20s long, and makes the regions, which function as the beams by being sandwiched between the slits 20s, long. Thus, it is possible to make the entirety of the region R, in which the slits 20s are provided, easily deformable. In the example of FIG. 2, the slits include four such slits.

The plurality of slits 20s may include one or more inner slits of which the one-side ends are positioned on the circumference of the first imaginary circle 20i and the other-side ends are separated from the circumference of the second imaginary circle 20o. The plurality of slits 20s may include outer slits of which the one-side ends are separated from the circumference of the first imaginary circle 20i and the other-side ends are positioned on the circumference of the second imaginary circle 20o. In the example of FIG. 2, the plurality of slits 20s include inner slits 21 and 22, and outer slits 23 and 24.

By the plurality of slits 20s including inner slits and outer slits, it is possible to secure on one surface of the strain generating body 20, regions on which strain gauges 100 are positioned. The number of inner slits and outer slits may be determined taking into consideration the number of strain gauges 100 to be positioned.

When the strain gauges 100 are sufficiently small in size, it is not necessary to provide inner slits and outer slits. That is, all of the plurality of slits 20s may be slits of which the one-side ends are positioned on the circumference of the first imaginary circle 20i and the other-side ends are positioned on the circumference of the second imaginary circle 20o.

In a case of providing inner slits and outer slits, one or more slits 20s may be provided between an inner slit and an outer slit. In this case, an imaginary straight line that connects the other-side end of the inner slit and the one-side end of the outer slit crosses one or more slits 20s. This makes it easier to secure regions on which the strain gauges 100 are positioned. In the example of FIG. 2, one slit 20s is provided between the other-side end of the inner slit 21 and the one-side end of the outer slit 23, and one slit 20s is provided between the other-side end of the inner slit 22 and the one-side end of the outer slit 24.

In the example of FIG. 2, eight slits 20s including the inner slits 21 and 22 and the outer slits 23 and 24 are provided in the strain generating body 20. In the example of FIG. 2, the eight slits 20s are two-fold symmetrical about the center of gravity G of the strain generating body 20.

When the strain generating body 20 has a circular shape as in the example of FIG. 2, it is preferable that the length of each slit 20s is 0.5 times or more and 1.2 times or less the diameter of the strain generating body 20. When the length of each slit 20s is in this range, it is possible to make the region R, in which the slits 20s are provided, easily deformable. Moreover, it is easy to adjust the resonance frequency of the strain generating body 20 within a range of 500 Hz or higher and 2 kHz or lower.

The width w of the slits 20s is preferably 0.025 mm or greater and 0.1 mm or less. When the width of the slits 20s is in this range, it is possible to make the region R, in which the slits 20s are provided, easily deformable. Moreover, it is easy to adjust the resonance frequency of the strain generating body 20 within a range of 500 Hz or higher and 2 KHz or lower.

The main frequency component of the pulse wave measured by the pulse wave sensor 1 is lower than 500 Hz. Thus, the resonance frequency of the strain generating body 20 is preferably 500 Hz or higher. As a result, it is possible to inhibit reduction in the measurement accuracy of the pulse wave sensor 1 due to the effects of the resonance frequency of the strain generating body 20. On the other hand, when the resonance frequency of the strain generating body 20 is higher than 2 kHz, a high-frequency noise may get superimposed on the signal measured by the pulse wave sensor 1. Thus, it is preferable that the resonance frequency of the strain generating body 20 is 500 Hz or higher and 2 kHz or lower. In order to improve the measurement accuracy of the pulse wave sensor 1, it is preferable that the resonance frequency of the strain generating body 20 is 800 Hz or higher and 1.5 kHz or lower, and more preferably 900 Hz or higher and 1.1 kHz or lower.

The strain generating body 20 may include a load portion 29 that projects from the upper surface 20m, which is the surface on the side to be in contact with a subject. The load portion 29 may be provided inside the first imaginary circle 20i. The load portion 29 may be provided over the entirety of the first imaginary circle 20i. The diameter of the load portion 29 may be approximately from ⅕ through ¼ the diameter of the strain generating body 20. The amount of projection of the load portion 29 measured from the upper surface 20m of the strain generating body 20 may be, for example, approximately 0.1 mm. By providing the strain generating body 20 with the load portion 29 that projects from the upper surface 20m, it is possible to facilitate transmission of a load corresponding to the pulse wave of a subject to the strain generating body 20.

The wire rod 30 is a cable for inputting/outputting electric signals between the pulse wave sensor 1 and the outside. The wire rod 30 may be a shield cable, a flexible board, or the like.

In the present disclosure, the strain gauges 100 are an example of a detector configured to detect a pulse wave. The strain gauges 100 are provided in the region R of the strain generating body 20. The strain gauges 100 may be provided on a lower surface 20n side of the strain generating body 20. Since the strain generating body 20 is a flat plate shape, the strain gauges can be easily pasted on. One or more strain gauges 100 may be provided, but in the present embodiment, four strain gauges 100 are provided. By providing four strain gauges 100, strain can be detected by a full bridge.

In the example of FIG. 3, two strain gauges 100 are positioned so as to face each other across a slit 20s that crosses an imaginary straight line connecting the other-side end of the inner slit 21 and the one-side end of the outer slit 23. Moreover, two strain gauges 100 are positioned so as to face each other across a slit 20s that crosses an imaginary straight line connecting the other-side end of the inner slit 22 and the one-side end of the outer slit 24.

Of the four strain gauges 100, two are positioned on a side (inner side) closer to the first imaginary circle 20i in the region R, and the other two are positioned on a side (outer side) closer to the second imaginary circle 20o in the region R. With such an arrangement, compressive and tensile forces can be effectively detected and a large output can be obtained by the full bridge.

FIG. 5 is a view illustrating a simulation result, and illustrates the magnitude of a strain of the strain generating body 20 when a load is applied to the center of the strain generating body 20 having the shape illustrated in FIG. 1 to FIG. 4. The simulation conditions are as follows. That is, the material of the strain generating body 20 is phosphor bronze, the diameter of the strain generating body 20 is 8.4 mm, the thickness t of the strain generating body 20 is 0.025 mm, the number of the slits 20s is 8, the length of the slits 20s is adjusted in a range of 0.5 times or more and 1.2 times or less the diameter of the strain generating body 20, and the width w of the slits 20s is 0.025 mm.

In FIG. 5, black represents portions almost unstrained, and gray represents portions largely strained. From FIG. 5, it can be seen that the region R in which the plurality of slits 20s were provided was largely strained. Here, the resonance frequency of the strain generating body 20 was approximately 1 kHz.

The pulse wave sensor 1 is used while being fixed to an arm of a subject such that the upper surface 20m side of the strain generating body 20 contacts the radial artery of the subject. When a load becomes applied to the strain generating body 20 in response to the pulse wave of the subject, the region R in which the plurality of slits 20s are provided elastically deforms as illustrated in FIG. 5, and the resistance values of the resistors of the strain gauges 100 positioned in the region R change along with this. That is, the pulse wave sensor 1 can detect a pulse wave based on changes in the resistance values of the resistors of the strain gauges 100 along with deformation of the region R of the strain generating body 20. For example, the pulse wave is output in the form of a periodical voltage change from a measuring circuit connected to electrodes of the strain gauges 100.

Hereinafter, the strain gauge 100 will be described.

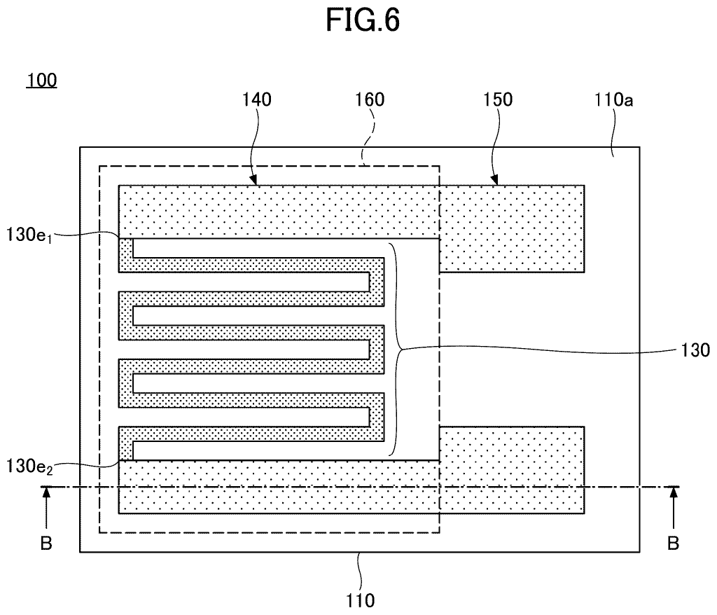

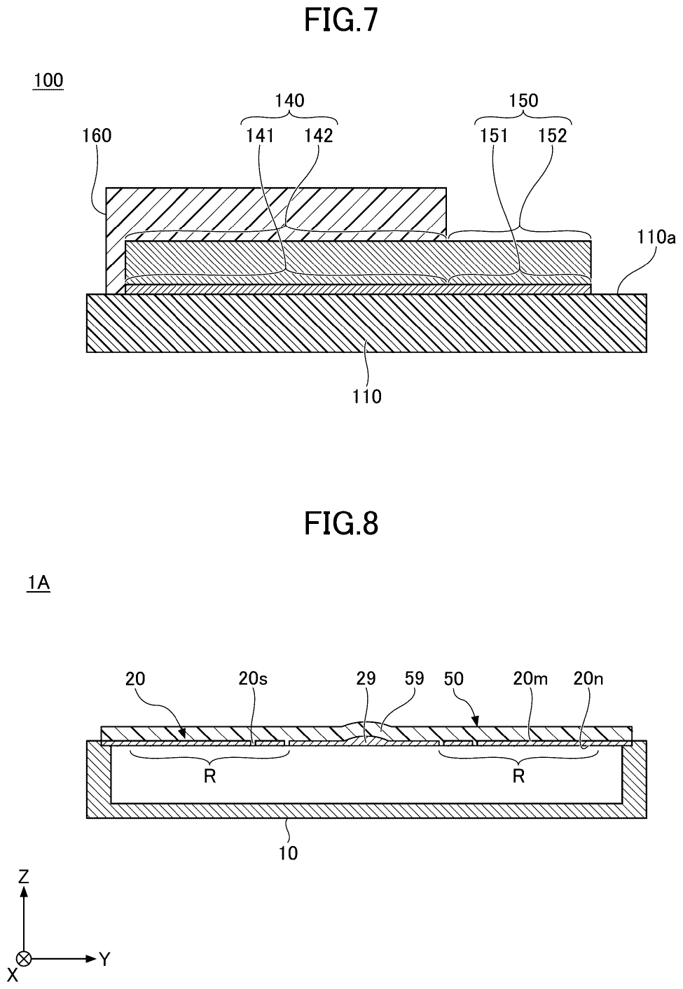

FIG. 6 is a plan view illustrating the strain gauge according to the first embodiment. FIG. 7 is a cross-sectional view illustrating the strain gauge according to the first embodiment, illustrating a cross-section along the line B-B of FIG. 6. Referring to FIGS. 16 and 17, the strain gauge 100 includes a substrate 110, a resistor 130, a wiring 140, an electrode 150, and a cover layer 160. In FIG. 6, only the outer edge of the cover layer 160 is illustrated as a dashed line for convenience. The cover layer 160 may be provided as necessary.

In FIGS. 6 and 7, for convenience, in the strain gauge 100, the side of the substrate 110 where the resistor 130 is provided is the upper side or one side, and the side where the resistor 130 is not provided is the lower side or the other side. In addition, the surface of each part on the side where the resistor 130 is provided is one surface or the upper surface, and the surface on the side where the resistor 130 is not provided is the other surface or the lower surface. However, the strain gauge 100 can be used in the reverse state or arranged at any angle. For example, in FIG. 3, the strain gauge 100 is attached to the strain generating body 20 in the state of being upside down from FIG. 7. That is, the substrate 110 in FIG. 7 is attached to the lower surface 20n of the strain generating body 20 with an adhesive or the like. The plan view refers to an object viewed in the direction normal to the upper surface 110a of the substrate 110, and the planar shape refers to the shape of the object viewed in the direction normal to the upper surface 110a of the substrate 110.

The substrate 110 is a member that serves as a base layer for forming the resistor 130 and the like, and the substrate 110 has flexibility. The thickness of the substrate 110 is not particularly limited and can be appropriately selected according to the purpose, but can be, for example, in a range from 5 μm to 500 μm. In particular, when the thickness of the substrate 110 is in a range from 5 μm to 200 μm, it is preferable in terms of the transmissibility of strain from the surface of the strain generating body bonded to the lower surface of the substrate 110 via an adhesive layer or the like and also in terms of the dimensional stability with respect to the environment. When the thickness of the substrate 110 is 10 μm or more, it is more preferable in terms of the insulating property.

The substrate 110 can be formed from, for example, an insulating resin film such as a polyimide (PI) resin, an epoxy resin, a polyetheretherketone (PEEK) resin, a polyethylene naphthalate (PEN) resin, a polyethylene terephthalate (PET) resin, a polyphenylene sulfide (PPS) resin, a liquid crystal polymer (LCP) resin, a polyolefin resin, and the like. The film refers to a member having a thickness of about 500 μm or less and having flexibility.

The term “formed from an insulating resin film” here does not prevent the substrate 110 from containing fillers, impurities or the like in the insulating resin film. The substrate 110 may be formed from, for example, an insulating resin film containing a filler such as silica, alumina, or the like.

Materials other than the resin for the substrate 110 include, for example, crystalline materials such as SiO₂, ZrO₂ (including YSZ), Si, Si₂N₃, Al₂O₃ (including sapphire), ZnO, perovskite-based ceramics (CaTiO₃, BaTiO₃), or the like, and further include amorphous glass or the like. Metals such as aluminum, aluminum alloy (duralumin), titanium, or the like may be used as materials for the substrate 110. In this case, for example, an insulating film is formed on the substrate 110 made of metal.

The resistor 130 is a thin film formed in a predetermined pattern on the substrate 110. The resistor 130 is a sensitive part that causes a resistance change when being strained. The resistor 130 may be formed directly on the upper surface 110a of the substrate 110, or through other layers on the upper surface 110a of the substrate 110. In FIG. 6, the resistor 130 is illustrated in a dark-colored pear-skin pattern for convenience.

The resistor 130 has a structure in which a plurality of thin and long portions are arranged at predetermined intervals with the longitudinal direction set in the same direction (i.e., the direction of the line B-B in FIG. 6), and the end portions of adjacent thin and long portions are connected on the alternate sides, and folded back in a zigzag manner as a whole. The longitudinal direction of the plurality of thin and long portions is a grid direction, and the direction perpendicular to the grid direction is a grid width direction (the direction perpendicular to the line B-B in FIG. 6).

The end portions, on the longitudinal-direction one side, of the two thin and long portions that are located on the outermost side in the grid width direction are bent in the grid width direction to form respective ends 130e₁ and 130e₂ in the grid width direction of the resistor 130. The respective ends 130e₁ and 130e₂ in the grid width direction of the resistor 130 are electrically connected to the electrodes 150 via the wiring 140. In other words, the wiring 140 electrically connects the respective ends 130e₁ and 130e₂ in the grid width direction of the resistor 130 with the respective electrodes 150.

The resistor 130 can be formed, for example, from a material containing chromium (Cr), a material containing nickel (Ni), or a material containing both Cr and Ni. That is, the resistor 130 can be formed from a material containing at least one of Cr and Ni. The material containing Cr includes, for example, a Cr mixed phase film. The material containing Ni includes, for example, copper nickel (Cu—Ni). The material containing both Cr and Ni includes, for example, nickel chromium (Ni—Cr).

Here, the Cr mixed phase film is a film in which Cr, CrN, Cr₂N, and the like are mixed. The Cr mixed phase film may contain unavoidable impurities such as chromium oxide and the like.

The thickness of the resistor 130 is not particularly limited and can be appropriately selected according to the purpose, but can be, for example, in a range from about 0.05 μm to 2 μm. In particular, when the thickness of the resistor 130 is 0.1 μm or more, it is preferable in that the crystallinity (for example, the crystallinity of α-Cr) of the crystals constituting the resistor 130 is improved. When the thickness of the resistor 130 is 1 um or less, it is more preferable in that film cracks caused by the internal stress of the film constituting the resistor 130 and warpage from the substrate 110 can be reduced. The width of the resistor 130 can be set to about in a range from 10 μm to 100 μm if the required specifications such as the resistance value and the lateral sensitivity are optimized and the wire breakage countermeasures are considered.

For example, when the resistor 130 is a Cr mixed phase film, the stability of the gauge characteristic can be improved by using alpha chromium (α-Cr), which is a stable crystalline phase, as the main component. Moreover, when the resistor 130 uses α-Cr as the main component, the gauge rate of the strain gauge 100 can be 10 or more, and the temperature coefficient of strain gauge rate TCS and the temperature coefficient of resistance TCR can be set within a range from −1000 ppm/° C. to +1000 ppm/° C. Here, the main component means that the target material occupies 50% by weight or more of the total material constituting the resistor, but from the viewpoint of improving the gauge characteristics, the resistor 130 preferably contains 80% by weight or more of α-Cr, and more preferably 90% by weight or more. α-Cr is Cr having a bcc structure (body-centered cubic lattice structure).

When the resistor 130 is a Cr mixed phase film, CrN and Cr₂N contained in the Cr mixed phase film are preferably 20% by weight or less. When CrN and Cr₂N contained in the Cr mixed phase film are 20% by weight or less, the lowering of the gauge ratio can be suppressed.

The proportion of Cr₂N in CrN and Cr₂N is preferably 80% by weight or more and less than 90% by weight, and more preferably 90% by weight or more and less than 95% by weight. When the proportion of Cr₂N in CrN and Cr₂N is 90% by weight or more and less than 95% by weight, the decrease in TCR (negative TCR) becomes more remarkable due to Cr₂N having semiconducting properties. In addition, by reducing ceramization, brittle fracture is reduced.

On the other hand, when a small amount of N₂ or atomic N is mixed or present in the film, N₂ Or atomic N escapes from the film to the outside of the film due to an external environment (for example, a high-temperature environment), resulting in a change in film stress. By creating chemically stable CrN, a stable strain gauge without generating the unstable N can be obtained.

The wiring 140 is formed on the substrate 110 and is electrically connected to the resistor 130 and the electrodes 150. The wiring 140 has a first metal layer 141 and a second metal layer 142 laminated on the upper surface of the first metal layer 141. The wiring 140 is not limited to a straight line and can be formed in any pattern. The wiring 140 can be any width and any length. In FIG. 6, for convenience, the wiring 140 and the electrodes 150 are illustrated in a lighter-colored pear-skin pattern than the resistor 130.

The electrode 150 is formed on the substrate 110 and is electrically connected to the resistor 130 via the wiring 140, and is formed in a substantially rectangular shape, for example, wider than the wiring 140. The electrode 150 is a pair of electrodes for outputting a change in the resistance value of the resistor 130 caused by strain to the outside, and a lead wire or the like for external connection is joined, for example.

The electrode 150 has a pair of first metal layers 151 and a second metal layer 152 laminated on the upper surface of each first metal layer 151. The first metal layers 151 are electrically connected to the ends 130e₁ and 130e₂ of the resistor 130 through the first metal layers 141 of the wiring 140. The first metal layer 151 is formed in a substantially rectangular shape in a plan view. The first metal layer 151 may be formed in the same width as the wiring 140.

Although the resistor 130, the first metal layer 141, and the first metal layer 151 are designated separately for convenience, they can be integrally formed of the same material in the same process. Accordingly, the resistor 130, the first metal layer 141, and the first metal layer 151 have substantially the same thickness. In addition, although the second metal layer 142 and the second metal layer 152 are designated separately for convenience, they can be integrally formed of the same material in the same process. Therefore, the thickness of the second metal layer 142 and the second metal layer 152 is substantially the same.

The second metal layers 142 and 152 are formed from a material having a lower resistance than the resistor 130 (the first metal layers 141 and 151). The material of the second metal layers 142 and 152 is not particularly limited if the material has a lower resistance than the resistor 130, and can be appropriately selected according to the purpose. For example, if the resistor 130 is a Cr mixed phase film, the material of the second metal layers 142 and 152 may include Cu, Ni, Al, Ag, Au, Pt, or an alloy of any of these metals, a compound of any of these metals, or a laminated film suitably laminated with any of these metals, alloys, or compounds. The thickness of the second metal layers 142 and 152 is not particularly limited and may be suitably selected according to the purpose, but may be, for example, in a range from 3 μm to 5 μm.

The second metal layers 142 and 152 may be formed on a part of the upper surfaces of the first metal layers 141 and 151, or may be formed on the entire upper surfaces of the first metal layers 141 and 151. One or more other metal layers may be laminated on the upper surface of the second metal layer 152. For example, the second metal layer 152 may be a copper layer, and a gold layer may be laminated on the upper surface of the copper layer. Alternatively, the second metal layer 152 may be a copper layer, and a palladium layer and a gold layer may be successively laminated on the upper surface of the copper layer. By making the top layer of the electrode 150 a gold layer, the solder wettability of the electrode 150 can be improved.

As described above, the wiring 140 has a structure in which the second metal layer 142 is laminated on the first metal layer 141 made of the same material as the resistor 130. Therefore, since the wiring 140 has a lower resistance than the resistor 130, it is possible to suppress the wiring 140 from functioning as a resistor. As a result, the strain detection accuracy by the resistor 130 can be improved.

In other words, by providing the wiring 140 with a lower resistance than the resistor 130, the substantially sensitive portion of the strain gauge 100 can be limited to a local region where the resistor 130 is formed. Therefore, the strain detection accuracy by the resistor 130 can be improved.

In particular, in a highly sensitive strain gauge with a gauge ratio of 10 or more using a Cr mixed phase film as the resistor 130, limiting the substantially sensitive portion to the local region where the resistor 130 is formed by making the wiring 140 less resistant than the resistor 130 has a remarkable effect on improving the strain detection accuracy. In addition, making the wiring 140 less resistant than the resistor 130 also has an effect on reducing the lateral sensitivity.

The cover layer 160 is formed on the substrate 110, covers the resistor 130 and the wiring 140, and exposes the electrode 150. A portion of the wiring 140 may be exposed from the cover layer 160. By providing the cover layer 160 covering the resistor 130 and the wiring 140, mechanical damage or the like can be prevented from occurring to the resistor 130 and the wiring 140. Further, by providing the cover layer 160, the resistor 130 and the wiring 140 can be protected from moisture or the like. The cover layer 160 may be provided so as to cover the entire portion excluding the electrode 150.

The cover layer 160 may be formed of, for example, an insulating resin such as a PI resin, an epoxy resin, a PEEK resin, a PEN resin, a PET resin, a PPS resin, or a composite resin (for example, silicone resin, polyolefin resin). The cover layer 160 may contain fillers or pigments. The thickness of the cover layer 160 is not particularly limited and can be appropriately selected according to the purpose, but can be, for example, about in a range from 2 μm to 30 μm.

In order to manufacture the strain gauge 100, first, a substrate 110 is prepared and a metal layer (designated as a metal layer A for convenience) is formed on the upper surface 110a of the substrate 110. The metal layer A is a layer which is finally patterned into the resistor 130, the first metal layer 141, and the first metal layer 151. Accordingly, the material and thickness of the metal layer A are the same as those of the resistor 130, the first metal layer 141, and the first metal layer 151.

The metal layer A can be formed by, for example, a magnetron sputtering method by using a raw material capable of forming the metal layer A as a target. Instead of the magnetron sputtering method, the metal layer A may be formed by a reactive sputtering method, a vapor deposition method, an arc ion plating method, a pulsed laser deposition method, or the like.

From the viewpoint of stabilizing the gauge characteristics, a functional layer with a predetermined thickness may preferably be formed on the upper surface 110a of the substrate 110 as a foundation layer by, for example, a conventional sputtering method before forming the metal layer A.

In the present invention, the functional layer refers to a layer having a function of promoting crystal growth of at least the metal layer A (resistor 130) which is an upper layer thereof. In addition, the functional layer preferably has a function of preventing oxidation of the metal layer A by oxygen and moisture contained in the substrate 110 and a function of improving adhesion between the substrate 110 and the metal layer A. The functional layer may further have other functions.

Since the insulating resin film constituting the substrate 110 contains oxygen and moisture, especially when the metal layer A contains Cr, Cr forms an auto-oxide film. Therefore, providing the functional layer to prevent oxidation of the metal layer A is effective.

The material of the functional layer is not particularly limited and can be suitably selected according to the purpose as long as the material has a function of promoting crystal growth of at least the metal layer A (resistor 130) which is an upper layer. Examples of the material of the functional layer include metals selected from the group consisting of chromium (Cr), titanium (Ti), vanadium (V), niobium (Nb), tantalum (Ta), nickel (Ni), yttrium (Y), zirconium (Zr), hafnium (Hf), silicon (Si), carbon (C), zinc (Zn), copper (Cu), bismuth (Bi), iron (Fe), molybdenum (Mo), tungsten (W), ruthenium (Ru), rhodium (Rh), rhenium (Re), osmium (Os), iridium (Ir), platinum (Pt), palladium (Pd), silver (Ag), gold (Au), cobalt (Co), manganese (Mn), and aluminum (Al). One type of the metal selected from the group or two or more types of the metal selected from the group can be used. An alloy of any metals selected from the group or a compound of any metals selected from the group may also be used.

Examples of the alloys include FeCr, TiAl, FeNi, NiCr, CrCu, and the like. Examples of the compounds include TiN, TaN, Si₃N₄, TiO₂, Ta₂O₅, SiO₂, and the like.

When the functional layer is formed from a conductive material such as a metal or alloy, the thickness of the functional layer is preferably 1/20 or less of the thickness of the resistor. In such a range, crystal growth of α-Cr can be promoted, and it is possible to prevent the strain detection sensitivity from being reduced due to a part of the current, which is flowing through the resistor, flowing to the functional layer.

When the functional layer is formed from a conductive material such as a metal or alloy, the thickness of the functional layer is preferably 1/50 or less of the thickness of the resistor. In such a range, the crystal growth of α-Cr can be promoted, and it is possible to further prevent the detection sensitivity of the strain from being lowered due to a part of the current, which is flowing through the resistor, flowing to the functional layer.

When the functional layer is formed from a conductive material such as a metal or alloy, the thickness of the functional layer is more preferably 1/100 or less of the thickness of the resistor. In such a range, it is possible to yet further prevent the detection sensitivity of the strain from being lowered due to a part of the current, which is flowing through the resistor, flowing to the functional layer.

When the functional layer is formed from an insulating material such as an oxide or nitride, the thickness of the functional layer is preferably in a range from 1 nm to 1 μm. In this range, crystal growth of α-Cr can be promoted, and the film can be easily formed without cracks in the functional layer.

When the functional layer is formed from an insulating material such as an oxide or nitride, the thickness of the functional layer is more preferably in a range from 1 nm to 0.8 μm. In this range, crystal growth of α-Cr can be promoted and the film can be formed more easily without cracks in the functional layer.

If the functional layer is formed from an insulating material such as an oxide or nitride, the thickness of the functional layer is more preferably in a range from 1 nm to 0.5 μm. In this range, crystal growth of α-Cr can be promoted and the film can be formed more easily without cracks in the functional layer.

The planar shape of the functional layer is patterned almost identically to the planar shape of the resistor illustrated in FIG. 6, for example. However, the planar shape of the functional layer is not limited to the case where it is almost identical to the planar shape of the resistor. In the case where the functional layer is formed from an insulating material, it is not necessary to pattern it in the same shape as the planar shape of the resistor. In this case, the functional layer may be formed in a solid shape at least in the region where the resistor is formed. Alternatively, the functional layer may be formed in a solid pattern over the entire upper surface of the substrate 110.

Further, when the functional layer is formed from an insulating material, the thickness of the functional layer is formed relatively thick, in a range from 50 nm to 1 μm, and in a solid pattern, which increases the thickness and surface area of the functional layer, allowing the heat generated by the resistor to dissipate to the substrate 110. As a result, in the strain gauge 100, a decrease in measurement accuracy due to self-heating of the resistor can be suppressed.

For example, the functional layer can be formed in vacuum by a conventional sputtering method in which argon (Ar) gas is introduced into a chamber by using a raw material capable of forming the functional layer as a target. By using the conventional sputtering method, the functional layer is formed while etching the upper surface 110a of the substrate 110 with Ar, so that the adhesion improvement effect can be obtained by minimizing the film formation amount of the functional layer.

However, this is an example of the film formation method of the functional layer, and the functional layer may be formed by other methods. For example, the adhesion improvement effect may be obtained by activating the upper surface 110a of the substrate 110 by plasma treatment using Ar or the like before the film formation of the functional layer, and then the functional layer may be formed in vacuum by a magnetron sputtering method.

The combination of the material of the functional layer and the material of the metal layer A is not particularly limited and can be appropriately selected according to the purpose, but, for example, a Cr mixed phase film in which α-chromium (α-Cr) is the main component can be formed as the metal layer A, using Ti as the functional layer.

In this case, for example, the metal layer A can be formed by a magnetron sputtering method in which Ar gas is introduced into the chamber by using a raw material capable of forming a Cr mixed phase film as a target. Alternatively, the metal layer A can be formed by a reactive sputtering method in which an appropriate amount of nitrogen gas is introduced into the chamber together with Ar gas by using pure Cr as a target. At this time, the proportion of CrN and Cr₂N contained in the Cr mixed phase film and the proportion of Cr₂N in CrN and Cr₂N can be adjusted by changing the amount of nitrogen gas introduced and the pressure (nitrogen partial pressure) or by adjusting the heating temperature by providing a heating step.

In these methods, the growth surface of the Cr mixed phase film is determined by the functional layer formed from Ti, and the Cr mixed phase film mainly composed of α-Cr, which is a stable crystal structure, can be formed. In addition, the Ti constituting the functional layer diffuses into the Cr mixed phase film, thereby improving the gauge characteristics. For example, the gauge rate of the strain gauge 100 may be 10 or more, and the temperature coefficient of strain gauge TCS and the temperature coefficient of resistance TCR may be within a range from −1000 ppm/° C. to +1000 ppm/° C. When the functional layer is formed from Ti, Ti or titanium nitride (TiN) may be included in the Cr mixed phase film.

In the case where the metal layer A is the Cr mixed phase film, the functional layer formed from Ti has a function of promoting crystal growth of the metal layer A, a function of preventing oxidation of the metal layer A by oxygen and moisture contained in the substrate 110, and a function of improving adhesion between the substrate 110 and the metal layer A. The same applies when Ta, Si, Al, and Fe are used instead of Ti as the functional layer.

Thus, by providing the functional layer in the lower layer of the metal layer A, the crystal growth of the metal layer A can be promoted, and the metal layer A composed of the stable crystal phase can be produced. As a result, the stability of the gauge characteristics can be improved in the strain gauge 100. Further, by diffusing the material constituting the functional layer into the metal layer A, the gauge characteristics can be improved in the strain gauge 100.

Next, the second metal layer 142 and the second metal layer 152 are formed on the upper surface of the metal layer A. The second metal layer 142 and the second metal layer 152 can be formed by, for example, a photolithography method.

Specifically, a seed layer is first formed to cover the upper surface of the metal layer A by, for example, a sputtering method or an electroless plating method. Next, a photosensitive resist is formed on the entire surface of the upper surface of the seed layer, and an opening is formed to expose an area for forming the second metal layer 142 and the second metal layer 152 by exposure and development. At this time, the pattern of the second metal layer 142 can be formed so as to be a desirable shape by adjusting the shape of the opening of the resist. For example, a dry film resist or the like can be used as the resist.

Next, for example, a second metal layer 142 and a second metal layer 152 are formed on the seed layer exposed in the opening by an electrolytic plating method using the seed layer as a power supply path. The electrolytic plating method is suitable in that the tact is high and a low-stress electrolytic plating layer can be formed as the second metal layer 142 and the second metal layer 152. By making the thick electrolytic plating layer low-stress, warping of the strain gauge 100 can be prevented. The second metal layer 142 and the second metal layer 152 may be formed by a non-electrolytic plating method.

Next, the resist is removed. The resist can be removed, for example, by dipping the resist material into a dissolvable solution.

A photosensitive resist is then formed on the entire surface of the upper surface of the seed layer, exposed and developed, and patterned into a planar shape similar to that of the resistor 130, the wiring 140, and the electrode 150 of FIG. 6. For example, a dry film resist and the like can be used as the resist. Then, using the resist as an etching mask, the metal layer A and the seed layer exposed from the resist are removed to form the planar resistor 130, the wiring 140, and the electrode 150 illustrated in FIG. 6.

For example, unnecessary portions of the metal layer A and the seed layer can be removed by wet etching. If a functional layer is formed under the metal layer A, the functional layer is patterned by etching into the planar shape illustrated in FIG. 6 in the same manner as the resistor 130, the wiring 140, and the electrode 150. At this point, the seed layer is formed on the resistor 130, the first metal layer 141, and the first metal layer 151.

Next, the second metal layer 142 and the second metal layer 152 are formed by using the second metal layer 142 and the second metal layer 152 as etching masks and removing unnecessary seed layers exposed from the second metal layer 142 and the second metal layer 152. The seed layers immediately below the second metal layer 142 and the second metal layer 152 remain. For example, the unnecessary seed layer can be removed by wet etching using an etchant in which the seed layer is etched and the functional layer, the resistor 130, the wiring 140, and the electrode 150 are not etched.

Then, if necessary, a cover layer 160 covering the resistor 130 and the wiring 140 and exposing the electrode 150 is provided on the upper surface 110a of the substrate 110 to form the strain gauge 100. For example, the cover layer 160 can be formed by laminating a semi-cured thermosetting insulating resin film on the upper surface 110a of the substrate 110 so as to cover the resistor 130 and the wiring 140 and expose the electrode 150, and curing the film by heating. The cover layer 160 can be prepared by coating the upper surface 110a of the substrate 110 with a liquid or paste thermosetting insulating resin covering the resistor 130 and the wiring 140 and exposing the electrode 150, and heating to cure the resin.

### Modified Examples of the First Embodiment

In a modified example of the first embodiment, an example of a pulse wave sensor including a resin layer covering a strain generating body will be set forth. In the modified example of the first embodiment, description of the same components as those in the embodiment described above may be omitted.

FIG. 8 is a cross-sectional view (part 1) illustrating the pulse wave sensor according to the modified example of the first embodiment. A pulse wave sensor 1A illustrated in FIG. 8 is different from the pulse wave sensor 1 (see FIG. 4 and the like) in that it includes a resin layer 50.

The resin layer 50 covers one surface of the strain generating body 20. In the present embodiment, the one surface is an upper surface 20m. The resin layer 50 may cover the entirety of the upper surface 20m of the strain generating body 20, or may cover a part of the upper surface 20m. The resin layer 50 is also formed on the slits 20s. Thus, the slits 20s are not exposed to the outside of the pulse wave sensor 1A. The resin layer 50 may intrude into the slits 20s and fill a part or the whole of each slit 20s.

As the resin layer 50, it is preferable to use a resin material having an elastic modulus of 10 GPa or lower. Examples of such a resin material include an epoxy resin, a silicone resin, and the like. When a resin material having an elastic modulus of 10 GPa or lower is used, the resin material constituting the resin layer 50 does not inhibit elastic deformation of the strain generating body 20 even when it intrudes into the gaps of the slits 20s.

As the resin layer 50, the resin material may be molded on the upper surface 20m of the strain generating body 20 using a molding die, or a resin film may be laminated on the upper surface 20m of the strain generating body 20. The thickness of the resin layer 50 may be, for example, approximately from 10 μm through 500 μm. Since the resin layer 50 is formed along the upper surface 20m of the strain generating body 20, a load portion 59 covering the load portion 29 is formed on the resin layer 50. The load portion 59 projects from the upper surface of the resin layer 50. The amount of projection of the load portion 59 measured from the upper surface of the resin layer 50 is, for example, approximately 0.1 mm.

Since the pulse wave sensor 1A includes the resin layer 50 covering the upper surface 20m of the strain generating body 20, the strain generating body 20 does not directly contact the skin of a subject. Thus, in a case where the strain generating body 20 is made of a metal and the subject using the pulse wave sensor has a predisposition to develop cutaneous inflammation due to a metal or metal allergy, use of the pulse wave sensor 1A allows the subject to avoid developing cutaneous inflammation due to the metal or metal allergy.

Given that the slits 20s are opened to the outside of the pulse wave sensor 1, dust, foreign matter, or the like gets stuck in the slits 20s and the strain generating body 20 may become unable to elastically deform. However, when it comes to the pulse wave sensor 1A, the resin layer 50 is also formed on the slits 20s, and the slits 20s are not exposed to the outside of the pulse wave sensor 1. Thus, no dust, foreign matter, or the like will get stuck in the slits 20s, and the pulse wave sensor 1A can perform more reliable and more stable pulse wave measurement.

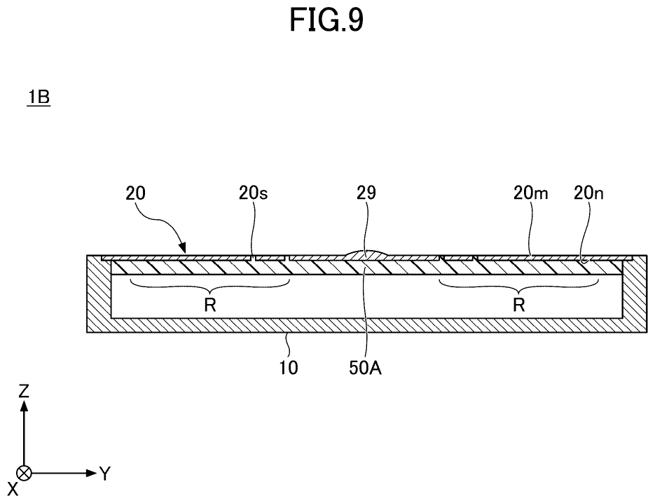

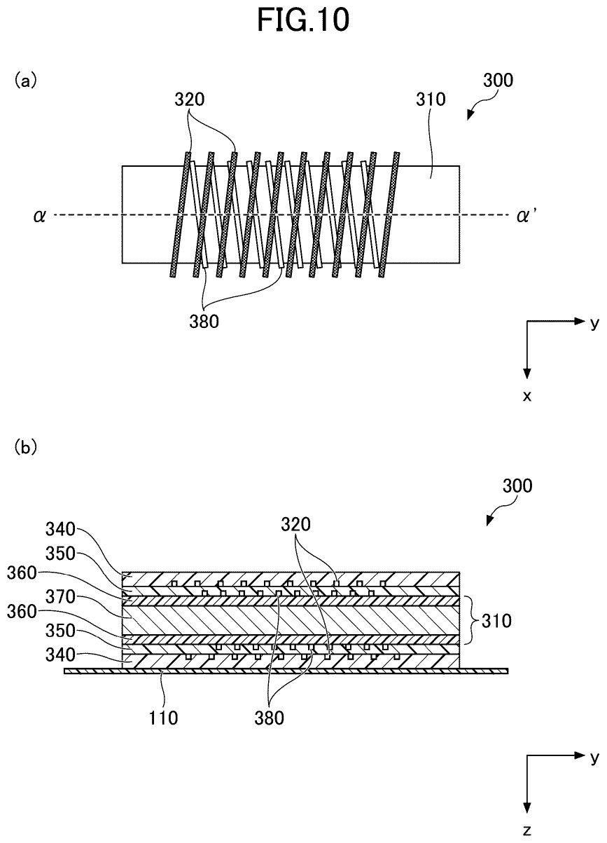

FIG. 9 is a cross-sectional view (part 2) illustrating a pulse wave sensor according to a modified example of the first embodiment. A pulse wave sensor 1B illustrated in FIG. 9 is different from the pulse wave sensor 1 (see FIG. 4 and the like) in that the it includes a resin layer 50A.

As in the pulse wave sensor 1B illustrated in FIG. 9, the resin layer 50A covering a lower surface 20n of the strain generating body 20 may be provided instead of providing the resin layer 50 covering the upper surface 20m of the strain generating body 20. The material and the thickness of the resin layer 50A are the same as those of the resin layer 50. In the pulse wave sensor 1B, the resin layer 50A intrudes into the slits 20s and fill the gaps of the slits 20s. Thus, no dust, foreign matter, or the like will get stuck in the slits 20s, and the pulse wave sensor 1B can perform more reliable and more stable pulse wave measurement. In addition, together with the cover layer 160, the resin layer 50A protects the strain gauge 100 from moisture and the like.

It is optional to provide the resin layer 50 covering the upper surface 20m of the strain generating body 20, and to also provide the resin layer 50A covering the lower surface 20n of the strain generating body 20.

### Second Embodiment

In the embodiment described above and the modified examples thereof, an example in which the detector according to the present disclosure is a strain gauge using a resistor has been described. That is, in the embodiment described above, a case in which the detector according to the present disclosure is an electric resistance-based metallic strain gauge has been described. However, the detector according to the present disclosure is not limited to a metallic strain gauge. For example, the detector according to the present disclosure may be a strain gauge configured to detect a magnetic change caused by a strain of a strain generating body (or a structure equivalent to the strain generating body), by using a detecting element included in the strain gauge.

Specifically, the detector according to the present disclosure may be a strain gauge including a detecting element utilizing the Villari effect (described below). The detector according to the present disclosure may be a strain gauge including a detecting element having a magnetic tunnel junction (described below) structure. In the following second embodiment, a strain gauge including a detecting element utilizing the Villari effect will be described. In the third embodiment, a strain gauge including a detecting element having a magnetic tunnel junction structure will be described.

In the embodiments of the present specification, the components having the same functions will be denoted by the same names and component numbers, and description of such components will not be repeated. The directions of the x-axis, the y-axis, and the z-axis in the drawings relating to the following embodiments are the same as the directions of the x-axis, the y-axis, and the z-axis indicated in FIG. 2 and FIG. 3.

FIG. 10 is a diagram illustrating an example of a detecting element 300 included in a strain gauge 100 according to the second embodiment. FIG. 10(a) is a plan view of the strain gauge 100 when pasted on the strain generating body 20 as illustrated in FIG. 1 to FIG. 3, and is a plan view in a case where the detecting element 300 is seen in a direction from its lower surface (i.e., the surface opposite to the pasting surface of the strain gauge 100) to its upper surface (i.e., the pasting surface of the strain gauge 100). Meanwhile, FIG. 10(b) is a cross-sectional view of the detecting element 300 illustrated in FIG. 10(a), taken along a surface α-α′. The views included in FIG. 10 do not illustrate wirings of the detecting element 300. However, the detecting element 300 may include a wiring for connecting a drive coil 320 described below and a power source with each other, and a wiring for transmitting a current detected by a sensing coil 380.

As illustrated in FIG. 10(a), the detecting element 300 includes the drive coil 320, the sensing coil 380, and a base layer 310. The sensing coil 380 is a coil including the base layer 310 as a core material. The drive coil 320 is a coil including the base layer 310 as a core material, and is wound on the outer side of the sensing coil. In this way, the drive coil 320 and the sensing coil 380 form a double structure in which the drive coil 320 is positioned on the outer side and the sensing coil 380 is positioned on the inner side. By winding the sensing coil 380 on the inner side of the drive coil 320, it is possible to apply an alternating magnetic field (described below) to the entirety of the sensing coil 380 uniformly. This improves the performance of the detecting element 300.

The drive coil 320 is a coil configured to generate a magnetic field. When an alternating current is supplied from a power source to the drive coil 320, the drive coil 320 generates an altermatic magnetic field around itself. The base layer 310 is a product obtained by coating an approximately flat plate-shaped metal plate (a base metal 370 described below) with an insulating layer (an insulating layer 360 described below). The metal plate of the base layer 310 is a magnetic body of the detecting element 300. The metal plate of the base layer 310 is magnetized by an alternating magnetic field generated by the drive coil 320. The sensing coil 380 is a coil configured to detect the magnetization magnitude of the base metal 370. The material of the drive coil 320 and the sensing coil 380 is preferably a conductive metal such as Cu, Ag, Al, Au, and the like, and alloys of these metals. The number of turns and the cross-sectional area of the drive coil 320 and the sensing coil 380 may be appropriately designed in accordance with the strain sensitivity required of the detecting element 300.

With reference to the cross-sectional view of FIG. 10(b), the detecting element 300 will be described in greater detail. Layers 340 to 360 described below are wound around the base metal 370 serving as the core material. Thus, in FIG. 10(b), it can be regarded that any layers denoted by the same component number are continuous around the base metal 370.

As described above, the detecting element 300 has a structure in which the sensing coil 380 and the drive coil 320 are wound around the base layer 310. The base layer 310 has a structure in which the base metal 370 is coated with the insulating layer 360. An insulating layer 350 is formed to surround the insulating layer 360. The insulating layer 350 is a layer including the sensing coil 380, and is a layer in which the gaps in the sensing coil 380 are filled with an insulating material. In addition, an insulating layer 340 is further formed to surround the insulating layer 350. The insulating layer 340 is a layer including the drive coil 320, and is a layer in which the gaps in the drive coil 320 are filled with an insulating material.

It is preferable to form the base metal 370 using, for example, a soft magnetic material such as a Fe-Si-Al-based alloy such as sendust and the like, and a Ni-Fe-based alloy such as permalloy and the like. It is preferable that the insulating layers 340, 350, and 360 are cured products of resists such as dry film, photosensitive polyimide, and the like that do not affect a magnetic field.

As illustrated in the cross-sectional view of FIG. 10(b), the pasting surface of the detecting element 300 is pasted on the substrate 110. The detecting element 300 may be a detecting element having a flat plate shape or a thin film shape as a whole. The detecting element 300 having a flat plate shape or a thin film shape is easier to paste on the substrate 110. The substrate 110 is then pasted on the strain generating body 20. The strain generating body 20 according to the present embodiment may be basically of the same configuration and material as those of the strain generating body 20 according to the first embodiment. However, it is more preferable that the strain generating body 20 is made of a nonmagnetic body. The strain generating body 20 according to the present embodiment may be made of, for example, a nonmagnetic stainless steel.

The detecting element 300 according to the present embodiment includes the base metal 370 serving as the magnetic body. When a current flows through the drive coil 320 and a magnetic field is generated, the base metal 370 is magnetized. When the strain generating body 20 deforms in this state, a strain is generated along with this. The strain is transmitted via the substrate 110 to apply a stress to the base metal 370. When a stress is applied to the base metal 370, the magnetic permeability of the base metal 370 changes in accordance with the stress, and the magnetization magnitude (magnetization degree) changes. The phenomenon, in which application of a stress to a magnetic body causes changes in the magnetic permeability and the magnetization magnitude of the magnetic body, is referred to as the “Villari effect”. According to the configuration of the detecting element 300, an alternating voltage corresponding to the magnetization magnitude of the base metal 370 is induced across the sensing coil 380, which is a pickup coil. Thus, based on the principle of the Villari effect, it is possible to calculate the stress applied to the base metal 370 (i.e., the straining degree of the substrate 110) based on the value of the alternating voltage. In the case of the example illustrated in FIG. 10(a), the grid direction of the detecting element 300 is the α-α′ direction in the drawing.

By this principle, the detecting element 300 can detect a strain received by the substrate 110 pasted on the strain generating body 20. That is, the detecting element 300 functions as the detecting element of the strain gauge 100.

The pulse wave sensor 1 according to the present embodiment is used while being fixed to an arm of a subject such that the upper surface 20m of the strain generating body 20 contacts the radial artery of the subject, like the pulse wave sensor 1 according to the first embodiment. When a load is applied to the strain generating body 20 in accordance with a pulse wave of the subject, the region R in which the plurality of slits 20s are provided elastically deforms as illustrated in FIG. 5, and the substrate 110 of the strain gauge 100 positioned in the region R strains along with this. The detecting element 300 of the strain gauge 100 can detect a magnetic change caused by this straining based on the above-described principle of the Villari effect.

The strain gauge 100 including the detecting element 300 according to the present embodiment can be provided at any positions specified in the first embodiment and the modified examples of the first embodiment. Thus, by using the detecting element 300 utilizing the Villari effect, it is possible to detect a strain of the strain generating body 20 as in the case of using a resistor strain gauge. Accordingly, the strain gauge 100 according to the present embodiment can achieve the same effect as that achieved by the strain gauges 100 according to the first embodiment and the modified examples of the first embodiment.

The substrate 110 is not an indispensable component of the detecting element 300. For example, the detecting element 300 may be used with its upper surface directly pasted on the strain generating body 20, instead of the substrate 110 being provided to the detecting element 300. When the detecting element 300 is pasted on the strain generating body 20 via no substrate 110, a stress is transmitted from the strain generating body 20 directly to the base metal 370 (and the insulating layers 340 to 360 covering it).

It is preferable that the drive coil 320 is wound as uniformly as possible on the outer side of the sensing coil 380 and over the entirety of the region where the sensing coil 380 exists. This enables more uniform application of an alternating magnetic field to an entire region of the base metal 370 where the sensing coil 380 exists. As a result, it is possible to more minutely detect Villari effect-based changes in the magnetization magnitude of the base metal 370. Thus, the performance of the detecting element 300 is improved.

The insulating layer 360 may be formed only on a part, not the entirety, of the base metal 370. For example, parts of the base metal 370 over which the sensing coil 380 and the drive coil 320 are wound may be covered with the insulating layer 360, the insulating layer 360 may be covered with the insulating layer 350 including the sensing coil 380, and the insulating layer 350 may be covered with the insulating layer 340 including the drive coil 320.

When the base metal 370 is an approximately flat plate shape, the insulating layer 360 may be formed to surround the base metal 370 only in the coil winding direction. That is, in FIG. 10(b), both ends of the base metal 370 in the y direction do not need to be covered with the insulating layer 360.

### Third Embodiment

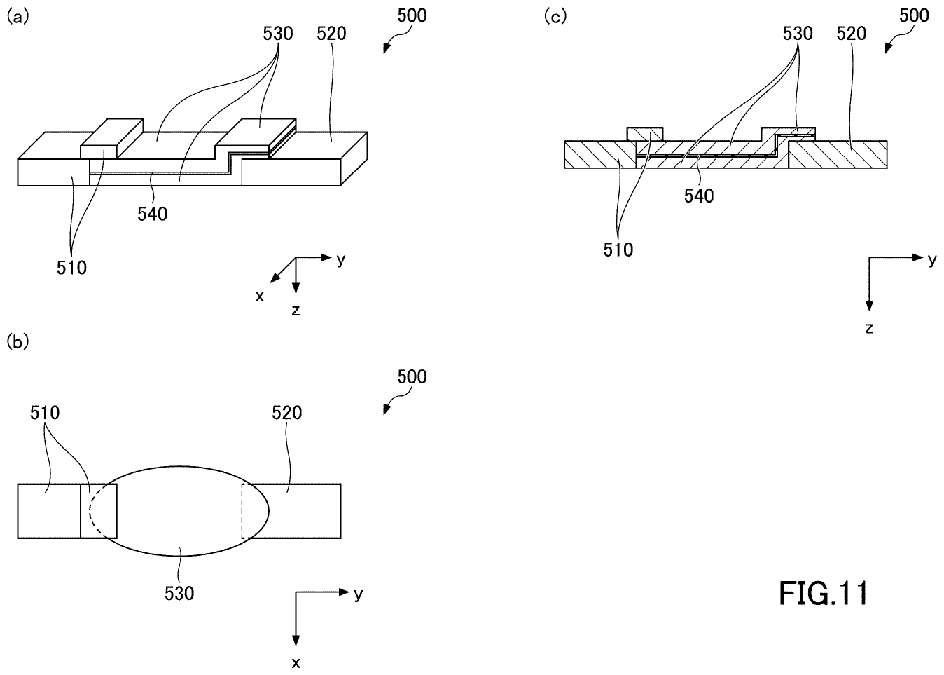

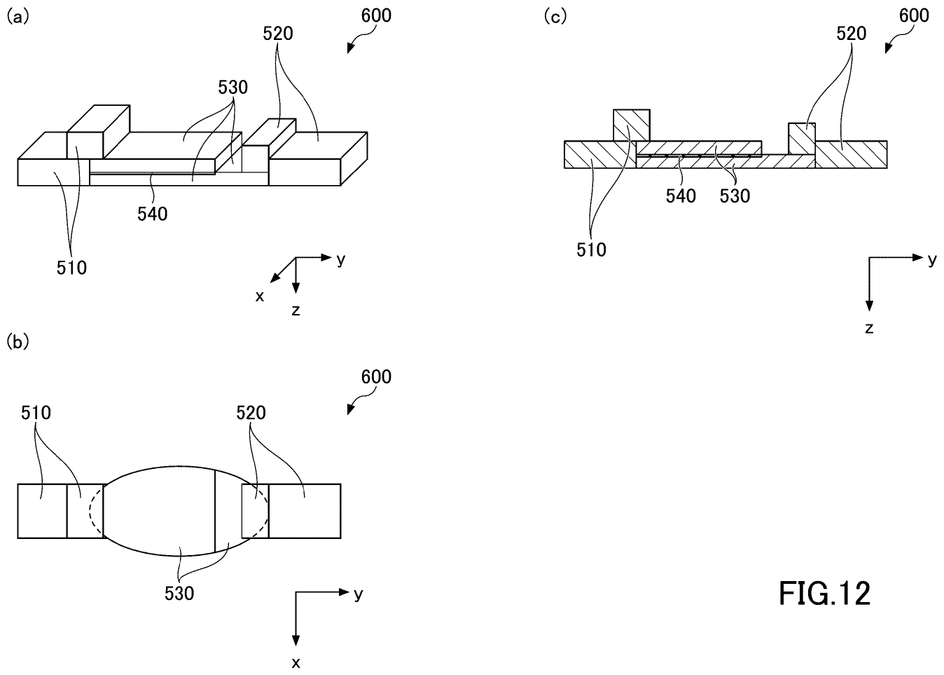

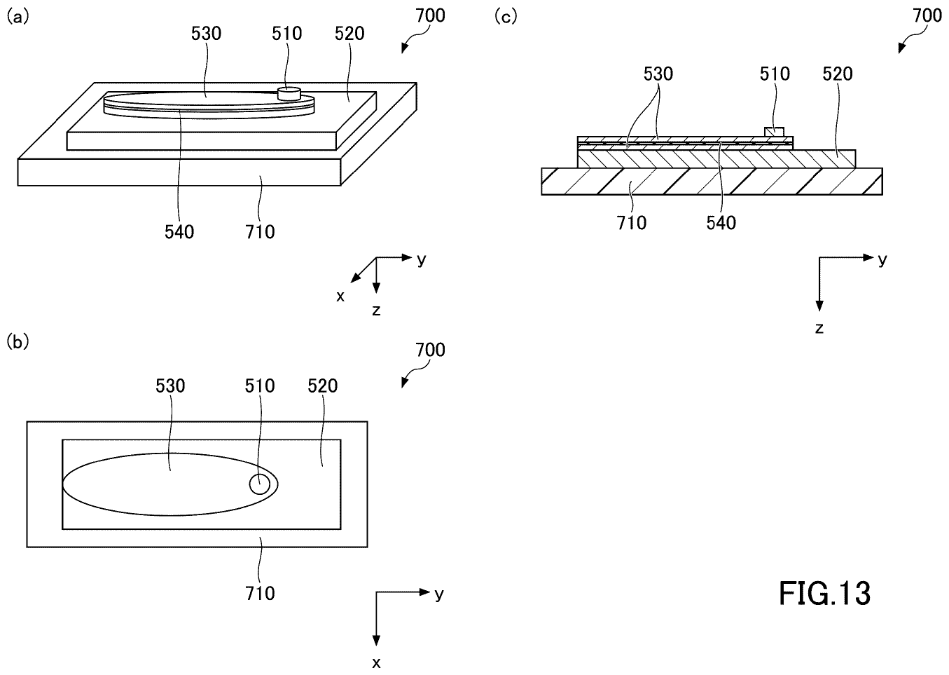

FIG. 11 is a view illustrating an example of a detecting element 500 included in a strain gauge 100 according to a third embodiment. FIG. 12 is a view illustrating another example of the detecting element according to the third embodiment. FIG. 13 is a view illustrating yet another example of the detecting element according to the third embodiment. The “upper side” and the “lower side” in the description with reference to FIG. 11 to FIG. 13 denote the same sides as the “upper side” and the “lower side” in FIGS. 1 to 4. That is, the positive side on the z-axis is the “upper side” in FIGS. 1 to 4, and the negative side on the z-axis is the “lower side” in FIGS. 1 to 4. FIGS. 11 to 13(a) are perspective views of detecting elements 500, 600, and 700, respectively. FIGS. 11 to 13(b) are plan views of the detecting elements 500, 600, and 700, looked down from the negative side to the positive side on the z-axis (i.e., from the lower side to the upper side in FIGS. 1 to 4), respectively. FIGS. 11 to 13(c) are cross-sectional views of the detecting elements 500, 600, and 700 along a surface parallel with the zy plane. The pasting surfaces of the detecting elements 500, 600, and 700 to be pasted on the substrate 110 are the upper-side flat surfaces (surfaces parallel with the xy plane). None of the views of FIGS. 11 to 13 illustrate wirings of the detecting element. However, the detecting elements 500, 600, and 700 may include a wiring for connecting an upstream electrode 510 described below and a power source with each other, and a wiring for connecting a downstream electrode 520 and a power source with each other.

As illustrated in FIGS. 11 to 13(a), the detecting elements 500, 600, and 700 each include an upstream electrode 510, a downstream electrode 520, magnetic films 530, and an insulating film 540. The insulating film 540 is sandwiched between the magnetic films 530 as illustrated. The magnetic films 530 and the insulating film 540 form a magnetic tunnel junction. That is, the detecting element 500 is a structure obtained by connecting electrodes to a magnetic tunnel junction structure.

A flexible substrate made of plastic film or the like may be provided on the further upper side of either or both of the upstream electrode 510 and the downstream electrode 520. The substrate may serve as the substrate 110.

The magnetic films 530 are magnetic nano-thin films. The insulating film 540 is an insulating nano-thin film. The materials of the magnetic films 530 and the insulating film 540 are not particularly limited so long as a magnetic tunnel junction structure can be formed. For example, cobalt-iron-boron, or 3d transition metal ferromagnetic materials such as Fe, Co, Ni, and the like, or alloys or the like containing them may be used as the magnetic films 530. Silicon oxide, silicon nitride, aluminum oxide, magnesium oxide, and the like may be used as the insulating film 540.

The upstream electrode 510 and the downstream electrode 520 are electrodes configured to apply a voltage across the magnetic tunnel junction structure. In the examples of FIGS. 11 to 13, the current flows from the upstream electrode 510 to the downstream electrode 520. In the case of, for example, FIG. 11(c), when a voltage is applied across the upstream electrode 510 and the downstream electrode 520, electrons flow from the lower magnetic film 530 to the upper magnetic film 530 beyond the insulating film 540. This is a phenomenon referred to as the “tunnel effect”, and the electric resistance against flowing of electrons via the insulating film 540 is referred to as the “tunnel resistance”. In the examples of FIGS. 11 to 13, end portions of the junctions between various parts of the electrodes are processed such that no current shorting the magnetic tunnel junction structure will flow.

When the detecting element 500 is strained via the substrate 110 or the like, a magnetic change occurs in the tunnel junction structure. More specifically, the upper and lower magnetic films 530 are magnetized in different directions. When the upper and lower magnetic films 530 are magnetized in different directions in this way, the tunnel resistance is greater than when the directions in which they are magnetized are parallel (tunnel magnetic resistance effect). Thus, in the detecting element 500 having the configuration described above, the current flowing between the electrodes becomes smaller in accordance with the magnitude of the strain of the detecting element 500 (more strictly, the magnetic tunnel junction part). That is, the greater the strain, the higher the electric resistance. In this way, the detecting element 500 can detect a strain based on the current value relative to the applied voltage. Thus, by pasting the detecting element 500 on the substrate 110, it is possible to measure a strain applied to the strain generating body 20.

The detecting element having the magnetic tunnel junction structure is not limited to the example illustrated in FIG. 11. For example, it is possible to employ the detecting elements 600 and 700 illustrated in FIG. 12 and FIG. 13. The detecting element 600 illustrated in FIG. 12 and the detecting element 700 illustrated in FIG. 13 are the same as the detecting element 500 in that they are formed of the upstream electrode 510, the downstream electrodes 520, the magnetic films 530, and the insulating film 540, and in the principle of detecting a strain by these components. The basic operations of the detecting elements 600 and 700 are also the same as that of the detecting element 500. The grid direction of the detecting elements 500, 600, and 700 corresponds to the y-axis direction (the y-axis positive direction and the y-axis negative direction) in FIG. 11 to FIG. 13. The detecting element 600 illustrated in FIG. 12 has a structure in which the upper magnetic film 530 and the lower magnetic film 530 are partially continuous, as illustrated. That is, the magnetic tunnel junction structure is formed only in a partial region of the magnetic films 530, and the tunnel magnetic resistance effect occurs in this structure. On the other hand, the detecting element 700 illustrated in FIG. 13 is pasted on the substrate 110 via a substrate 710. As illustrated in FIG. 11 to FIG. 13, the designs of the detecting elements may be appropriately changed in accordance with requisites such as size, durability, detecting target stress magnitude, and the like, as long as such design changes are not beyond the principle described above.

Basically, the strain generating body 20 according to the present embodiment may be of the same configuration and the same material as those of the strain generating body 20 according to the first embodiment. However, it is desirable that the strain generating body 20 is made of a nonmagnetic body. The strain generating body 20 according to the present embodiment may be manufactured using, for example, a nonmagnetic stainless steel as the material. The detecting elements 500, 600, and 700 may be an approximately flat plate shape such as a film shape as a whole element. Thus, the detecting element 500 can be easily pasted on the substrate 110. The detecting elements 500, 600, and 700 may have a structure, such as a drive coil structure or the like, for applying a weak magnetic field to the magnetic tunnel junction structure part. By applying a magnetic field to the magnetic tunnel junction structure part, it is possible to measure the tunnel magnetic resistance effect described above more stably, making it possible to detect a strain stably.

The “upstream electrode” and the “downstream electrode” of the detecting elements 500, 600, and 700 are the names used for convenience, and the current flowing direction may be reversed. That is, the current may be designed to flow from the downstream electrode 520 to the upstream electrode 510 in the detecting elements 500, 600, and 700 illustrated in FIG. 11 to FIG. 13.

The pulse wave sensor 1 according to the present embodiment is used while being fixed to an arm of a subject such that the upper surface 20m of the strain generating body 20 contacts the radial artery of the subject, like the pulse wave sensor 1 according to the first embodiment. When a load is applied to the strain generating body 20 in accordance with a pulse wave of the subject, the region R in which the plurality of slits 20s are provided elastically deforms as illustrated in FIG. 5, and the substrate 110 of the strain gauge 100 provided in the region R strains along with this. The detecting element 500, 600, or 700 of the strain gauge 100 can detect a magnetic change caused by this straining based on the above-described principle of the Villari effect.

The strain gauges 100 including the detecting elements 500, 600, and 700 according to the present embodiment can be provided at any positions specified in the first embodiment and the modified examples of the first embodiment. Thus, by using the detecting elements 500, 600, and 700 utilizing the magnetic tunnel effect, it is possible to detect a strain of the strain generating body 20, as in the case of using a resistor strain gauge. Accordingly, the strain gauge 100 according to the present embodiment can achieve the same effect as that achieved by the strain gauges 100 according to the first embodiment and the modified examples of the first embodiment.

Also for the detecting elements 500, 600, and 700, the substrate 110 is not an indispensable component. For example, the detecting elements 500, 600, and 700 may be used with their upper surface directly pasted on the strain generating body 20 or 20A.

It is preferable that the drive coil 320 is wound as uniformly as possible on the outer side of the sensing coil 380 and over the entirety of the region where the sensing coil 380 exists. This enables more uniform application of an alternating magnetic field to an entire region of the base metal 370 where the sensing coil 380 exists. As a result, it is possible to more minutely detect Villari effect-based changes in the magnetization magnitude of the base metal 370. Thus, the performance of the detecting element 300 is improved.

### Fourth Embodiment

The detector according to the present disclosure may be a semiconductor strain gauge, a capacitive pressure sensor, or an optical fiber strain gauge. The detector according to the present disclosure may be a mechanical pressure sensor, a vibrating pressure sensor, or a piezoelectric pressure sensor. Principles of various strain gauges and pressure sensors will be described below.

### (Semiconductor Strain Gauge)

A semiconductor strain gauge is a strain gauge configured to detect a strain by utilizing the piezoresistive effect of a semiconductor. That is, a semiconductor strain gauge is a strain gauge using a semiconductor as a strain detecting element.

It is known that when a stress is applied to a semiconductor, the crystal lattice of the semiconductor strains and the number and mobility of carriers in the semiconductor change, resulting in a change in the electric resistance. The semiconductor strain gauge can be used while being directly pasted on the strain generating body 20 like the electric resistance-based metallic strain gauge. In this case, when the strain generating body 20 expands or contracts, the pasted semiconductor (more specifically, the crystal lattice of the semiconductor) strains, to change the electric resistance. Thus, by measuring the electric resistance, it is possible to determine the amount of the strain of the strain generating body 20.

Moreover, the semiconductor strain gauge can be configured as a strain sensor having a diaphragm structure. In this case, for example, the strain sensor includes a nonmetallic diaphragm (or a product including an electrically insulating layer on a metallic diaphragm), and a semiconductor (for example, a semiconductor made of a silicon thin film) formed on the diaphragm. In such a structure including a diaphragm, when the diaphragm strains due to a perpendicular stress applied to the diaphragm, the electric resistance of the semiconductor changes. Thus, by measuring the electric resistance, it is possible to determine the amount of the strain of the diaphragm (and the amount of the strain of the strain generating body 20, as a natural course of events).

### (Capacitive Pressure Sensor)

A capacitive pressure sensor is a pressure sensor configured to measure a pressure applied to a diaphragm in the form of a change in the capacitance across a pair of electrodes. That is, the capacitive pressure sensor is a pressure sensor using a pair of electrodes as detecting elements. For example, the capacitive pressure sensor includes a diaphragm as a movable electrode, and one or more fixed electrodes. For example, the diaphragm is made of silicon containing an impurity (i.e., silicon functioning as a conductor) or the like.

When a pressure is applied to the diaphragm, the diaphragm is displaced, to change the distance between the fixed electrodes and the movable electrode. It is known that the capacitance across the electrodes is determined in accordance with the distance between the electrodes, provided that the permittivity of the medium between the electrodes and the electrode areas are constant. Thus, by measuring the capacitance, it is possible to determine the amount of the displacement of the diaphragm (i.e., the magnitude of the pressure).

### (Optical Fiber Strain Gauge)

An optical fiber strain gauge is a strain gauge configured to detect a strain by using an optical fiber including Fiber Bragg Gratings (FBGs). That is, an optical fiber strain gauge is a strain gauge using an optical fiber as a strain detecting element. The FBGs are diffraction gratings configured to reflect light in a different manner from the other parts of the optical fiber. The individual gratings are formed at constant intervals. When the optical fiber strains and becomes extended, the grating interval of the FBGs is increased. Therefore, a wavelength change occurs between incident light (e.g., laser light) coming into the optical fiber and its reflected light. Moreover, when the optical fiber strains and becomes contracted, the grating interval of the FBGs is reduced. Therefore, a wavelength change occurs between incident light (e.g., laser light) coming into the fiber and its reflected light.

By pasting the optical fiber having this property on the strain generating body 20 and measuring the wavelength spectrum of reflected light in the optical fiber, it is possible to determine the amount of the strain of the optical fiber (i.e., the amount of the strain of the strain generating body 20). The optical fiber strain gauge may be a strain gauge configured to determine the amount of a strain of the optical fiber based on a change in the frequency of Brillouin-scattered light occurring in the optical fiber.

### (Mechanical Pressure Sensor)

A mechanical pressure sensor is a sensor configured to determine a pressure applied to a mechanical structure by measuring the amount of displacement of the structure. For example, the mechanical pressure sensor includes a spring or a bent tube, and is configured to measure the amount of expansion or contraction of the spring or the amount of expansion or contraction of the bent tube. The amount of their expansion or contraction (i.e., the amount of displacement) changes in accordance with the magnitude of the pressure applied to the spring or the bent tube. Thus, by measuring the amount of expansion or contraction, it is possible to determine the pressure applied to the spring or the bent tube. The shape and the size of the spring or the bent tube may be appropriately determined in accordance with the size and the shape of the target to which the mechanical pressure sensor is attached.

### (Vibrating Pressure Sensor)

A vibrating pressure sensor is a sensor configured to detect a pressure by utilizing a phenomenon that the natural frequency of an elastic beam changes in accordance with a pressure (an axial load) that occurs along the axis of the elastic beam. The vibrating pressure sensor can be used while being directly pasted on the strain generating body 20, like the electric resistance-based metallic strain gauge. Moreover, for example, the vibrating pressure sensor may be a pressure sensor including a diaphragm formed on a substrate and a beam-like vibrator formed on the surface of the diaphragm.

In any case, when the strain generating body 20 strains, the straining pressure is directly or indirectly transmitted to the vibrator, to apply an axial load on the vibrator. The natural frequency of the vibrator changes in accordance with the axial load. Thus, by measuring the natural frequency of the vibrator, it is possible to determine the magnitude of the pressure on the strain generating body 20.

### (Piezoelectric Pressure Sensor)

A piezoelectric pressure sensor is a sensor including a piezoelectric element (also referred to as a piezo element), and configured to detect a pressure by utilizing the characteristics of the piezoelectric element. The piezoelectric element has a characteristic of generating an electromotive force corresponding to a force applied thereto, when it deforms (strains) in response to the application of the force. The piezoelectric element also has a characteristic of generating a force corresponding to a voltage applied thereto and undergoing expansion or contraction, in response to the application of the voltage.

The piezoelectric pressure sensor can determine the force applied to the piezoelectric element (i.e., the amount of a strain of the piezoelectric element) by measuring the electromotive force of the piezoelectric element. Thus, by pasting the piezoelectric pressure sensor on the strain generating body 20, it is possible to determine the amount of a strain of the strain generating body 20.

As described above, also in a case of using the semiconductor strain gauge, the capacitive pressure sensor, the optical fiber strain gauge, the mechanical pressure sensor, the vibrating pressure sensor, and the piezoelectric pressure sensor, it is possible to achieve the same effect as that achieved by the strain gauge 100 according to the first embodiment and the modified examples of the first embodiment.

Preferred embodiments and the like have been described above. However, the pulse wave sensor according to the present disclosure is not limited to the embodiments and modified examples described above. For example, various modifications and substitutions are applicable to the pulse wave sensor according to the embodiments and the like described above, without departing from the scope specified in the claims.

The present international application claims priority to Japanese Patent Application No. 2022-030926 filed Mar. 1, 2022, Japanese Patent Application No. 2022-129701 filed Aug. 16, 2022, and Japanese Patent Application No. 2023-013171 filed Jan. 31, 2023. The entire contents of Japanese Patent Application No. 2022-030926, Japanese Patent Application No. 2022-129701, and Japanese Patent Application No. 2023-013171 are incorporated herein by reference.

### REFERENCE SIGNS LIST

1,1A,1B: pulse wave sensor, 10: housing, 20: strain generating body, 20i: first imaginary circle, 20m: upper surface, 20n: lower surface, 20o: second imaginary circle, 20s: slit, 21,22: inner slit, 23,24: outer slit, 29,59: load portion, 30: wire rod, 50,50A: resin layer, 100: strain gauge, 110: substrate, 110a: upper surface, 130: resistor, 140: wiring, 150: electrode, 160: cover layer, 130e₁, 130e₂: end, 300,500,600,700: detecting element, 310: base layer, 320: drive coil, 340,350,360: insulating layer, 370: base metal, 380: sensing coil, 510: upstream electrode, 520: downstream electrode, 530: magnetic film, 540: insulating film, 710: substrate

1. A pulse wave sensor, comprising:
a strain generating body including a plurality of slits each having an elongated shape and bending in a same direction; and
a strain gauge provided on the strain generating body and including a Cr mixed phase film as a resistor,
wherein the plurality of slits are varied in a distance from a center of gravity of the strain generating body to one-side ends of the slits and in a distance from the center of gravity of the strain generating body to the other-side ends of the slits, and
the pulse wave sensor is configured to detect a pulse wave based on a change in a resistance value of the resistor in response to a deformation of the strain generating body.

2. The pulse wave sensor according to claim 1,
wherein the plurality of slits are positioned in a region between a circumference of a first circle centered on the center of gravity of the strain generating body and a circumference of a second circle centered on the center of gravity of the strain generating body and having a diameter larger than that of the first circle, and
the plurality of slits include two or more slits, of which the one-side ends are positioned on the circumference of the first circle and the other-side ends are positioned on the circumference of the second circle.

3. The pulse wave sensor according to claim 2,
wherein the plurality of slits include:
one or more inner slits, of which the one-side ends are positioned on the circumference of the first circle and the other-side ends are separated from the circumference of the second circle; and
one or more outer slits, of which the one-side ends are separated from the circumference of the first circle and the other-side ends are positioned on the circumference of the second circle.

4. The pulse wave sensor according to claim 3,
wherein a straight line connecting the other-side end of the inner slit and the one-side end of the outer slit crosses one slit of the slits.

5. The pulse wave sensor according to claim 4,
wherein two strain gauges are positioned so as to face each other across the slit crossed by the straight line, each of the two strain gauges being the strain gauge.

6. The pulse wave sensor according to claim 2,
wherein the plurality of slits are two-fold symmetrical about the center of gravity of the strain generating body.

7. The pulse wave sensor according to claim 2,
wherein the strain generating body includes a load portion inside the first circle, the load portion projecting from a surface of the strain generating body on a side to be in contact with a subject.

8. The pulse wave sensor according to claim 2,
wherein the strain generating body is a circular shape having a diameter larger than that of the second circle.

9. The pulse wave sensor according to claim 8,
wherein a length of each of the slits is 0.5 times or more and 1.2 times or less the diameter of the strain generating body.

10. The pulse wave sensor according to claim 1,
wherein a width of each of the slits is 0.025 mm or greater and 0.1 mm or less.

11. The pulse wave sensor according to claim 1,
wherein a thickness of the strain generating body is 0.025 mm or greater and 0.2 mm or less.

12. The pulse wave sensor according to claim 1,
wherein a resonance frequency of the strain generating body is 900 Hz or higher and 1.1 kHz or lower.

13. The pulse wave sensor according to claim 1, further comprising:
a resin layer covering one surface of the strain generating body,
wherein the strain gauge is provided on the other surface of the strain generating body positioned on an opposite side to the one surface.

14. The pulse wave sensor according to claim 13, further comprising:
a second resin layer provided on the other surface of the strain generating body and covering the strain gauge.

15. A pulse wave sensor, comprising:
a strain generating body including a plurality of slits each having an elongated shape and bending in a same direction; and
a detector provided on the strain generating body,
wherein the plurality of slits are varied in a distance from a center of gravity of the strain generating body to one-side ends of the slits and in a distance from the center of gravity of the strain generating body to the other-side ends of the slits,
the detector is configured to detect either or both of a deformation of the strain generating body and a pressure applied to the strain generating body, and
the pulse wave sensor is configured to detect a pulse wave based on a change in either or both of the deformation and the pressure detected by the detector.

16.-28. (canceled)

29. The pulse wave sensor according to claim 15,
wherein the detector includes a detecting element configured to detect a magnetic change caused by a deformation of the strain generating body.

30. The pulse wave sensor according to claim 29,
wherein the detecting element includes a magnetic body, and
the detecting element is a detecting element configured to detect a change in a magnetization magnitude of the magnetic body in response to a pressure being applied to the magnetic body due to the deformation of the strain generating body.

31. The pulse wave sensor according to claim 29,
wherein the detecting element includes a structure of a magnetic tunnel junction in which an insulating film is sandwiched between magnetic films, and
the detecting element is a detecting element configured to detect a magnetic change caused in the structure due to the deformation of the strain generating body.

32.-34. (canceled)
